(19) United States
(12) Patent Application Publication
(10) Pub. No.: US 2020/0069462 A1
(43) Pub. Date: Mar. 5, 2020

(54) MICRO-STUD FORMULATION PREPARING AND OCULOPATHY TREATING AND PREVENTING

(71) Applicants: Yunxiang LIU, San Diego, CA (US); Eric LIU, Medford, MA (US)

(72) Inventors: Yunxiang LIU, San Diego, CA (US); Eric LIU, Medford, MA (US)

(21) Appl. No.: 16/554,070

(22) Filed: Aug. 28, 2019

Related U.S. Application Data
(60) Provisional application No. 62/725,075, filed on Aug. 30, 2018.

(51) Int. Cl. A61F 9/00; A61K 47/32; A61B 17/02; A61K 31/27; A61K 31/4425; A61K 31/14; A61K 31/407; A61K 31/55; A61K 9/00

(52) U.S. Cl. CPC A61F 9/0008; A61K 47/32; A61B 17/0231; A61K 31/27; A61B 2017/00004; A61K 31/14; A61K 31/407; A61K 31/55; A61K 9/0048; A61K 31/4425

(57) ABSTRACT

Disclosed are an ophthalmic micro-stud formulation, a process for preparing the same and a method for treating and preventing oculopathy with the same. The ophthalmic micro-stud formulation comprises a drug-loading part as a head of the micro-stud, a lubrication part as a bottom of the micro-stud, and a soluble carrier material, wherein the lubrication part is attached to the soluble carrier material.

### CROSS-REFERENCE TO RELATED APPLICATIONS

The present application claims priority to and the benefit of U.S. Provisional Application No. 62/725,075, filed Aug. 30, 2018, the disclosure of which is hereby incorporated by reference in its entirety.

### FIELD

The present disclosure generally relates to the medical field. In particular, the present disclosure relates to the ophthalmic field.

### BACKGROUND

Eyelids consist of thin folds of skin, muscle, and connective tissue. The eyelids protect the eyes and spread tears over the front of the eyes. The inside of the eyelids are lined with the conjunctiva of the eyelid (the palpebral conjunctiva), and the outside of the lids are covered with the body's thinnest skin. Some common eyelid disorders include the following: stye, blepharitis, chalazion, entropion, ectropion, eyelid edema, eyelid tumors and myasthenia gravis.

The main treatment for eyelid disorders is currently by administration oral preparation or eyedrops. However, unwanted systemic side effects can often occur with administration oral preparation, including nausea/vomiting, diarrhea, stomach pain, increased salivation and tearing, irregular heartbeat, restlessness, anxiety, muscle twitching or tremor, blurred vision, and difficulty breathing. In addition, dosing with oral preparation or eyedrops is multiple times a day, which can negatively impact quality of life and reduce compliance.

### SUMMARY

In one aspect, the disclosure relates to an ophthalmic micro-stud formulation, comprising an active pharmaceutical ingredient, a biodegradable material and a pharmaceutically acceptable excipient.

In another aspect, the present disclosure relates to an ophthalmic micro-stud formulation, comprising a drug-loading part as a head of the micro-stud, a lubrication part as a bottom of the micro-stud, and a soluble carrier material, wherein the lubrication part is attached to the soluble carrier material.

In yet another aspect, the present disclosure relates to a process for preparing an ophthalmic micro-stud formulation, comprising preparing a micro-stud via 3DP (three dimensional printing), mold-based hot embossing, injection molding, mold-based centrifuging, mold-based vacuum, mold-based photopolymerization, droplet-born air blowing, or stretching photolithography, wherein the ophthalmic micro-stud formulation comprises an active pharmaceutical ingredient, a biodegradable material and a pharmaceutically acceptable excipient.

In still another aspect, the present disclosure relates to a process for preparing an ophthalmic micro-stud formulation, comprising preparing a micro-stud via 3DP (three dimensional printing), mold-based hot embossing, injection molding, mold-based centrifuging, mold-based vacuum, mold-based photopolymerization, droplet-born air blowing, or stretching photolithography, wherein the ophthalmic micro-stud formulation comprises a drug-loading part as a head of the micro-stud, a lubrication part as a bottom of the micro-stud, and a soluble carrier material, wherein the lubrication part is attached to the soluble carrier material.

In still yet another aspect, the present disclosure relates to a method for treating and preventing oculopathy, comprising administering an ophthalmic micro-stud formulation to a palpebral conjunctiva superior to a superior tarsal border of an affected eye of a subject in need thereof, wherein the ophthalmic micro-stud formulation comprises an active pharmaceutical ingredient, a biodegradable material and a pharmaceutically acceptable excipient.

In still yet another aspect, the present disclosure relates to a method for treating and preventing oculopathy, comprising administering an ophthalmic micro-stud formulation to a palpebral conjunctiva superior to a superior tarsal border of an affected eye of a subject in need thereof, wherein the ophthalmic micro-stud formulation comprises a drug-loading part as a head of the micro-stud, a lubrication part as a bottom of the micro-stud, and a soluble carrier material, wherein the lubrication part is attached to the soluble carrier material.

### DETAILED DESCRIPTION

In the following description, certain specific details are included to provide a thorough understanding for various disclosed embodiments. One skilled in the relevant art, however, will recognize that the embodiments may be practiced without one or more these specific details, or with other methods, components, materials, etc.

Unless the context required otherwise, throughout the specification and claims which follows, the term “comprise” and variations thereof, such as “comprises” and “comprising” are to be construed in an open, inclusive sense, which is as “include, but not limited to”.

Reference throughout this specification to “one embodiment”, or “an embodiment”, or “in another embodiment”, or “in some embodiments” means that a particular referent feature, structure or characteristic described in connection with the embodiments is included in at least one embodiment. Therefore, the appearance of the phrases “in one embodiment”, or “in the embodiment”, or “in another embodiment”, or “in some embodiments” in various places throughout this specification are not necessarily all referring to the same embodiment. Moreover, the particular features, structures or characteristics may be combined in any suitable manner in one or more embodiments.

It should be noted that, as used in this specification and the appended claims, the singular forms “a”, “an” and “the” include plural referents unless the context clearly stated otherwise. Therefore, for example, a reaction comprising “a pharmaceutically acceptable excipient” comprises one pharmaceutically acceptable excipient, two or more pharmaceutically acceptable excipients.

In one aspect, the disclosure relates to an ophthalmic micro-stud formulation, comprising an active pharmaceutical ingredient, a biodegradable material and a pharmaceutically acceptable excipient.

The exemplary biodegradable materials that can be used in the present disclosure include, but not limited to, PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide) and PHB (poly-β-hydroxybutyrate).

The exemplary pharmaceutically acceptable excipients that can be used in the present disclosure include, but not limited to, lubricating compositions.

In some embodiments of the present disclosure, the ophthalmic micro-stud formulation has one micro-stud.

In some embodiments of the present disclosure, the ophthalmic micro-stud formulation has an array of micro-studs.

The exemplary micro-studs that can be used in the present disclosure include, but not limited to, elevated cylindrical micro-studs, cone-shaped micro-studs, cube-like micro-studs and rectangle-like micro-studs.

In some embodiments of the present disclosure, the micro-stud is attached to a carrier material.

In some embodiments of the present disclosure, the micro-studs are attached to a carrier material.

The exemplary carrier materials that can be used in the present disclosure include, but not limited to, a thin and flexible carrier sheet and a device having a substrate and an operating unit on one side of the substrate.

In some embodiments of the present disclosure, the micro-studs are detachably attached to the other side of the substrate.

In some embodiments of the present disclosure, the micro-studs are fixedly attached to the other side of the substrate.

The exemplary shapes of the operating unit that can be used in the present disclosure can be cylindrical shape, prismatic shape and other irregular shapes.

The exemplary shapes of the surface of the substrate can be plane shape, curved shape and flexible shape that can be adjusted per se in accordance with the curve of the contact surface.

In some embodiments of the present disclosure, the thin and flexible carrier sheet can be made by high-molecular-weight polymer, metal or silicon.

The exemplary high-molecular-weight polymers that can be used in the present disclosure include, but not limited to, natural polymers, derivatives of natural polymers, synthetic polymers and derivatives of synthetic polymers.

The exemplary high-molecular-weight polymers that can be used in the present disclosure include, but not limited to, hydrophobic polymer materials.

The exemplary high-molecular-weight polymers that can be used in the present disclosure include, but not limited to, acrylic resins, polyurethane, propylene glycol alginate, polyetherimide, high density polyethylene and polycarbonate.

In some embodiments of the present disclosure, the carrier material has a long diameter of not more than about 22 mm.

In some embodiments of the present disclosure, the carrier material has a short diameter of not more than about 4 mm.

In some embodiments of the present disclosure, the height of the micro-stud is not more than about 2 mm.

In some embodiments of the present disclosure, the width of the micro-stud is not more than about 2 mm.

In some embodiments of the present disclosure, the height of each micro-stud in the array is not more than about 2 mm.

In some embodiments of the present disclosure, the width of each micro-stud in the array is not more than about 2 mm.

In some embodiments of the present disclosure, the micro-stud comprises a drug-loading part as a head of the micro-stud, and a lubrication part as a bottom of the micro-stud.

In some embodiments of the present disclosure, the bottom of the micro-stud is in the proximity of a carrier material.

In some embodiments of the present disclosure, the bottom of each micro-stud is in the proximity of a carrier material.

In some embodiments of the present disclosure, the ophthalmic micro-stud formulation comprises a drug-loading layer and a carrier material.

In some embodiments of the present disclosure, the ophthalmic micro-stud formulation comprises a drug-loading layer, a lubricating composition and a carrier material, wherein the lubricating composition locates between the drug-loading layer and the carrier material.

The exemplary lubricating compositions that can be used in the present disclosure include, but not limited to, natural polymers, derivatives of natural polymers, synthetic polymers and derivatives of synthetic polymers.

The exemplary natural polymers that can be used in the present disclosure include, but not limited to, cellulose ethers, natural gums, starches and modified products of starches.

The exemplary cellulose ethers that can be used in the present disclosure include, but not limited to, methyl cellulose, carboxymethyl cellulose, sodium carboxymethylcellulose, ethyl cellulose, hydroxyethyl cellulose, methyl 2-hydroxyethyl cellulose, hydroxypropyl cellulose and hypromellose.

The exemplary natural gums that can be used in the present disclosure include, but not limited to, acacia senegal, acacia gum, guar gum, locust bean gum, tamarind polysaccharide gum, sesbania gum, linseed gum, gleditsia sinensis lam gum, pectin, abelmoschus manihot gums, carrageenan, agar, sodium alginate, potassium alginate, gelatin, chitin, xanthan gum, β-cyclodextrin, polydextrose, gellan gum and sodium hyaluronate.

The exemplary starches and modified products of starches that can be used in the present disclosure include, but not limited to, starch, carboxymethyl starch sodium, sodium starch phosphate, hydroxypropyl starch ether, acetylated distarch phosphate, hydroxypropyl distarch phosphate, phosphated distarch phosphate, sodium starch octenyl succinate, oxystarch, acetylated distarch adipate, acid modified starch, aluminum starch octenylbutanedioate and starch acetate.

The exemplary synthetic polymers that can be used in the present disclosure include, but not limited to, acrylic resins, polyurethane and propylene glycol alginate.

The exemplary acrylic resins that can be used in the present disclosure include, but not limited to, methacrylic acid copolymer, ethyl acrylic acid copolymer, propyl acrylic copolymer and butyl acrylic acid copolymer.

The exemplary methacrylic acid copolymers that can be used in the present disclosure include, but not limited to, polymers copolymerized with methyl methacrylate or methacrylic acid and one or more of the following monomers in any ratio, wherein the exemplary monomers include, but not limited to, methyl methacrylate, methacrylic acid, and butyl methacrylate. 2-(dimethylamino) ethyl methacrylate, ethyl acrylate, 2-(trimethylammonio)ethyl 2-methylpropenoate, methyl acrylate and 2-(dimethylamino)ethyl methacrylate.

The exemplary methacrylic acid copolymers that can be used in the present disclosure include, but not limited to, butyl methacrylate/2-(dimethylamino)ethyl methacrylate/methyl methacrylate (1:2:1) copolymer, methacrylic acid/ethyl acrylate (1:1) copolymer, methacrylic acid and methyl methacrylate (1:1) copolymer, methacrylic acid/methyl methacrylate (1:2) copolymer, ethyl propenoate groups/methyl 2-methylpropenoate groups/2-(trimethylammonio)ethyl 2-methylpropenoate groups copolymer (1:2:0.2), ethyl propenoate groups/methyl 2-methylpropenoate groups/2-(trimethylammonio)ethyl 2-methylpropenoate groups copolymer (1:2:0.1), ethyl acrylate/methyl methacrylate (2:1) copolymer, and methacrylic acid/methyl acrylate/methyl methacrylate (1:1:1) copolymer.

The exemplary lubricating compositions that can be used in the present disclosure include, but not limited to, gels and hydrophilic polymers.

The exemplary active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

In some embodiments of the present disclosure, the ophthalmic micro-stud formulation further comprises a lubricant.

The exemplary lubricants that can be used in the present disclosure include, but not limited to, diacetylated monoglycerides, glycol propylene, polysorbate 80, PEG (polyethylene glycol), triethyl citrate, glycerol triacetate, glycerides and fumaric acid.

In some embodiments of the present disclosure, the ophthalmic micro-stud formulation further comprises a plasticizer.

In some embodiments of the present disclosure, the ophthalmic micro-stud formulation comprises an active pharmaceutical ingredient, a biodegradable material and a pharmaceutically acceptable excipient, wherein the active pharmaceutical ingredient comprises a first active pharmaceutical ingredient and a second active pharmaceutical ingredient.

The exemplary first active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

The exemplary second active pharmaceutical ingredients that can be used in the present disclosure include, but not limited to, hemostatics and coagulants.

The exemplary hemostatics and coagulants that can be used in the present disclosure include, but not limited to, active ingredient acting on blood vessels, antifibrinolytic, thrombin, active ingredient promoting activation of blood coagulation factor, thromboplastin and fibrinogen.

The exemplary active ingredients acting on blood vessels that can be used in the present disclosure include, but not limited to, pituitrin, norepinephrine, epinephrine, somatostatin, carbazochrome and etamsylate.

The exemplary antifibrinolytics that can be used in the present disclosure include, but not limited to, tranexamic acid, aminomethylbenzoic acid, glycine, diacetaminophen and trasylol. The exemplary thrombins that can be used in the present disclosure include, but not limited to, prothrombin complex and hemocoagulase.

The exemplary active ingredients promoting activation of coagulation factor that can be used in the present disclosure include, but not limited to, coagulation factor I, coagulation factor II, coagulation factor III, coagulation factor IV, coagulation factor V, coagulation factor VII, coagulation factor VIII, coagulation factor IX, coagulation factor X, coagulation factor XII, coagulation factor XIII, Fitzgerald factor, von Willebrand factor, phylloquinone, menaquinone-n and protamine sulfate.

In some embodiments of the present disclosure, the ophthalmic micro-stud formulation can be hollow.

In some embodiments of the present disclosure, the ophthalmic hollow micro-stud formulation can be made by high-molecular-weight polymers, metal, silicon or drug-loaded high-molecular-weight polymers.

In some embodiments of the present disclosure, the ophthalmic micro-stud formulation can be sustained-released in vivo in about one week.

In some embodiments of the present disclosure, the ophthalmic micro-stud formulation can be sustained-released in vivo in about two weeks.

In some embodiments of the present disclosure, the ophthalmic micro-stud formulation can be sustained-released in vivo in about one to three months.

In some embodiments of the present disclosure, the ophthalmic micro-stud formulation can be sustained-released in vivo in about six months.

In some embodiments of the present disclosure, the ophthalmic micro-stud formulation can be sustained-released in vivo in more than about six months.

In another aspect, the present disclosure relates to an ophthalmic micro-stud formulation, comprising a drug-loading part as a head of the micro-stud, a lubrication part as a bottom of the micro-stud, and a soluble carrier material, wherein the lubrication part is attached to the soluble carrier material.

In some embodiments of the present disclosure, the lubrication part is a drug-free part.

In some embodiments of the present disclosure, the lubrication part comprises a gel material, hyaluronic acid (HA) or a mixture thereof.

The exemplary gel materials that can be used in the present disclosure include, but not limited to, carbomer, poloxamer, calcium polycarbophil, polyethylene oxide and polyethylene glycol.

The exemplary carbomers that can be used in the present disclosure have viscosity from about 4,000 to about 100,000 in a water dispersion system with a mass concentration of 0.5-1%.

The exemplary carbomers that can be used in the present disclosure include, but not limited to, Carbopol® 971P, Carbopol® 974 P, Carbopol® 980, Carbopol® 981, Carbopol® 5984, Carbopol® ETD 2020, Carbopol® Ultrez 10, Carbopol® 934, Carbopol® 934P, Carbopol® 940, Carbopol® 941, Carbopol® 1342.

The exemplary poloxamers that can be used in the present disclosure have a molecular weight of from about 3,000 to about 15,000.

The exemplary poloxamers that can be used in the present disclosure include, but not limited to, poloxamer 188, poloxamer 237, poloxamer 338, poloxamer 407.

The exemplary polyethylene oxides that can be used in the present disclosure have a molecular weight of from about 100,000 to about 7,000,000. The exemplary polyethylene oxides that can be used in the present disclosure include, but not limited to, Polyox™ WSR N10 NF, Polyox™ WSR N80 NF, Polyox™ WSR N750 NF, Polyox™ WSR 205 NF, Polyox™ WSR 1105 NF, Polyox™ WSR N12K NF, Polyox™ WSR N60K NF, Polyox™ WSR 301 NF, Polyox™ WSR Coagulant NF and Polyox™ WSR 303 NF.

The exemplary polyethylene glycols (PEGs) that can be used in the present disclosure have a molecular weight of from about 500 to about 10,000.

The exemplary polyethylene oxides that can be used in the present disclosure include, but not limited to, PEG 540, PEG 600, PEG 900, PEG 1000, PEG 1450, PEG 1540, PEG 2000, PEG 3000, PEG 3350, PEG 4000, PEG 4600 and PEG 8000.

In some embodiments of the present disclosure, the soluble carrier layer comprises polyvinyl pyrrolidone.

In some embodiments of the present disclosure, the drug-loading part comprises an active pharmaceutical ingredient (API), a biodegradable material and a pharmaceutically acceptable excipient.

The exemplary biodegradable materials that can be used in the present disclosure include, but not limited to, PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide) and PHB (poly-β-hydroxybutyrate).

In some embodiments of the present disclosure, the ophthalmic micro-stud formulation has one micro-stud.

In some embodiments of the present disclosure, the ophthalmic micro-stud formulation has an array of micro-studs.

The exemplary micro-studs that can be used in the present disclosure include, but not limited to, elevated cylindrical micro-studs, cone-shaped micro-studs, cube-like micro-studs and rectangle-like micro-studs.

In some embodiments of the present disclosure, the carrier material has a long diameter of not more than about 22 mm.

In some embodiments of the present disclosure, the carrier material has a short diameter of not more than about 4 mm.

In some embodiments of the present disclosure, the height of the micro-stud is not more than about 2 mm.

In some embodiments of the present disclosure, the width of the stud is not more than about 2 mm.

In some embodiments of the present disclosure, the height of each micro-stud in the array is not more than about 2 mm.

In some embodiments of the present disclosure, the width of each micro-stud in the array is not more than about 2 mm.

In some embodiments of the present disclosure, the bottom of each micro-stud is in the proximity of the soluble carrier material.

In some embodiments of the present disclosure, the micro-stud is hollow.

In some embodiments of the present disclosure, the active pharmaceutical ingredient (API) comprises a first active pharmaceutical ingredient and a second active pharmaceutical ingredient.

The exemplary first active pharmaceutical ingredients that can be used in the present disclosure include, but not limited to, neostigmine bromide, neostigmine, pyridostigmine, edrophonium chloride, ambenonium chloride, physostigmine, demecarium bromide and galantamine.

The exemplary second active pharmaceutical ingredients that can be used in the present disclosure include, but not limited to, hemostatics and coagulants.

The exemplary hemostatics and coagulants that can be used in the present disclosure include, but not limited to, active ingredient acting on blood vessels, antifibrinolytic, thrombin, active ingredient promoting activation of blood coagulation factor, thromboplastin and fibrinogen.

The exemplary active ingredients acting on blood vessels that can be used in the present disclosure include, but not limited to, pituitrin, norepinephrine, epinephrine, somatostatin, carbazochrome and etamsylate.

The exemplary antifibrinolytics that can be used in the present disclosure include, but not limited to, tranexamic acid, aminomethylbenzoic acid, glycine, diacetaminophen and trasylol.

The exemplary thrombins that can be used in the present disclosure include, but not limited to, prothrombin complex and hemocoagulase.

The exemplary active ingredients promoting activation of coagulation factor that can be used in the present disclosure include, but not limited to, coagulation factor I, coagulation factor II, coagulation factor III, coagulation factor IV, coagulation factor V, coagulation factor VII, coagulation factor VIII, coagulation factor IX, coagulation factor X, coagulation factor XII, coagulation factor XIII, Fitzgerald factor, von Willebrand factor, phylloquinone, menaquinone-n and protamine sulfate.

In some embodiments of the present disclosure, the backing layer of the micro-studs can soften after contacting a small amount of an aqueous solution within a short time. This characteristic is greatly useful for administrating the ophthalmic micro-stud formulation of the present disclosure to palpebral conjunctiva that has little body fluid.

In some embodiments of the present disclosure, the ophthalmic micro-stud formulation can be put onto palpebral conjunctiva and soften to conform to curve of palpebral conjunctiva in a short time.

In yet another aspect, the present disclosure relates to a process for preparing the ophthalmic micro-stud formulation comprising preparing a micro-stud via 3DP (three dimensional printing), mold-based hot embossing, mold-based centrifuging, mold-based vacuum, inj ection molding, mold-based photopolymerization, droplet-born air blowing, or stretching photolithography, wherein the ophthalmic micro-stud formulation comprises an active pharmaceutical ingredient, a biodegradable material and a pharmaceutically acceptable excipient.

The exemplary 3DP (three dimensional printing) that can be used in the present disclosure includes, but not limited to, fused deposition modelling, direct metal laser-sintering, electron beam melting, selective laser sintering, selective laser melting, selective heat sintering, stereo lithography appearance, digital light processing, polyjet, multi jet printing, continuous liquid interface production, two-photon polymerization, 3DP (three dimensional printing) and gluing, binder jetting, color jet printing, nanoparticle jetting, laminated object manufacturing, laser engineered net shaping, multi-jet fusion, plaster-based 3DP (three dimensional printing), laser cladding forming and syringe-pump-based 3DP (three dimensional printing).

In still another aspect, the present disclosure relates to a process for preparing the ophthalmic micro-stud formulation comprising preparing a micro-stud via 3DP (three dimensional printing), mold-based hot embossing, mold-based centrifuging, mold-based vacuum, inj ection molding, mold-based photopolymerization, droplet-born air blowing, or stretching photolithography, wherein the ophthalmic micro-stud formulation comprises a drug-loading part as a head of the micro-stud, a lubrication part as a bottom of the micro-stud, and a soluble carrier material, wherein the lubrication part is attached to the soluble carrier material.

The exemplary 3DP (three dimensional printing) that can be used in the present disclosure includes, but not limited to, fused deposition modelling, direct metal laser-sintering, electron beam melting, selective laser sintering, selective laser melting, selective heat sintering, stereo lithography appearance, digital light processing, polyjet, multi jet printing, continuous liquid interface production, two-photon polymerization, 3DP (three dimensional printing) and gluing, binder jetting, color jet printing, nanoparticle jetting, laminated object manufacturing, laser engineered net shaping, multi jet fusion, plaster-based 3DP (three dimensional printing), laser cladding forming and syringe-pump-based 3DP (three dimensional printing).

In still yet another aspect, the present disclosure relates to a method for treating and preventing oculopathy, comprising administering the ophthalmic micro-stud formulation to a palpebral conjunctiva superior to a superior tarsal border of an affected eye of a subject in need thereof, wherein the ophthalmic micro-stud formulation comprises an active pharmaceutical ingredient, a biodegradable material and a pharmaceutically acceptable excipient.

The exemplary oculopathy that can be treated or prevented by the method of the present disclosure includes, but not limited to, ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the method comprises removing the carrier material.

In some embodiments of the present disclosure, the method comprises applying a bandage contact lens to protect the eye for a short duration of time.

In some embodiments of the present disclosure, the method comprises prior to everting an upper eyelid to expose a palpebral conjunctiva cleaning the surgical area in a standard, sterile, oculoplastic and ophthalmic manner with betadine° swabs.

In some embodiments of the present disclosure, the method comprises after applying the ophthalmic micro-stud formulation cleaning the surgical area with sterile saline.

In some embodiments of the present disclosure, the method for treating and preventing oculopathy, comprising:

applying a drop of ophthalmic topical anesthetic to the affected eye;

cleaning the surgical area in a standard, sterile, oculoplastic and ophthalmic manner with betadine® swabs;

everting an upper eyelid of the affected eye to expose a palpebral conjunctiva;

applying an ophthalmic micro-stud formulation to the palpebral conjunctiva superior to a superior tarsal border;

optionally removing the carrier material;

cleaning the surgical area with sterile saline;

applying a bandage contact lens to protect the eye for a short duration of time;

applying appropriate topical antibiotic coverage during the interim; and

returning the eyelid to its normal anatomic position,

wherein the ophthalmic micro-stud formulation comprises an active pharmaceutical ingredient, a biodegradable material and a pharmaceutically acceptable excipient.

The treatment methods and ophthalmic micro-stud described in the present disclosure have the advantages of targeted, local administration and minimization of systemic side effects.

In still yet another aspect, the present disclosure relates to a method for treating and preventing oculopathy, comprising administering the ophthalmic micro-stud formulation to a palpebral conjunctiva superior to a superior tarsal border of an affected eye of a subject in need thereof, wherein the ophthalmic micro-stud formulation comprises a drug-loading part as a head of the micro-stud, a lubrication part as a bottom of the micro-stud, and a soluble carrier material, wherein the lubrication part is attached to the soluble carrier material.

The exemplary oculopathy that can be treated or prevented by the method of the present disclosure includes, but not limited to, ocular myasthenia gravis (OMG), blepharospasm, dermatolysis palpebrarum, involutional, myogenic, neurogenic, and congenital ptosis, trichiasis and eyelid tumors.

In some embodiments of the present disclosure, the method comprises everting an upper eyelid to expose a palpebral conjunctiva.

In some embodiments of the present disclosure, the method comprises applying a drop of ophthalmic topical anesthetic to the affected eye.

In some embodiments of the present disclosure, the method comprises applying a bandage contact lens to protect the eye for a short duration of time.

In some embodiments of the present disclosure, the method comprises prior to everting an upper eyelid to expose a palpebral conjunctiva cleaning the surgical area in a standard, sterile, oculoplastic and ophthalmic manner with betadine® swabs.

In some embodiments of the present disclosure, the ophthalmic micro-stud formulation is inserted into the conjunctiva so that it implants into the Mueller's muscle/levator muscle and aponeurosis superior to the superior tarsal border across the horizontal width of the eyelid. It should extend into as close to the fornix as possible.

In some embodiments of the present disclosure, the method comprises after applying the ophthalmic micro-stud formulation cleaning the surgical area with sterile saline.

In some embodiments of the present disclosure, the method for treating and preventing oculopathy, comprising:

applying a drop of ophthalmic topical anesthetic to the affected eye;

cleaning the surgical area in a standard, sterile, oculoplastic and ophthalmic manner with betadine® swabs;

everting an upper eyelid of the affected eye to expose a palpebral conjunctiva;

applying an ophthalmic micro-stud formulation to the palpebral conjunctiva superior to a superior tarsal border;

cleaning the surgical area with sterile saline;

applying a bandage contact lens to protect the eye for a short duration of time;

applying appropriate topical antibiotic coverage during the interim; and

returning the eyelid to its normal anatomic position,

wherein the ophthalmic micro-stud formulation comprises a drug-loading part as a head of the micro-stud, a lubrication part as a bottom of the micro-stud, and a soluble carrier material, wherein the lubrication part is attached to the soluble carrier material.

The treatment methods and ophthalmic micro-stud described in the present disclosure have the advantages of targeted, local administration and minimization of systemic side effects.

### EXAMPLES

Although anyone skilled in the art is capable of preparing the formulations of the present disclosure according to the general techniques disclosed above, more specific details on synthetic techniques for formulations of the present disclosure are provided elsewhere in this specification for convenience. Again, all reagents and reaction conditions employed in synthesis are known to those skilled in the art and are available from ordinary commercial sources.

### Materials and Experiment Equipment:

Neostigmine bromide: Hubei Guangao Biotechnology Co., Ltd./GA20181205

PLGA: Jinan Daigang Biomaterial Co., Ltd./20181112804

PCL: Jinan Daigang Biomaterial Co., Ltd./2018101210

PLA: Jinan Daigang Biomaterial Co., Ltd./2018120605

ACRYPOL 971P: Corel Pharma Chem/44217010

Polyvinyl pyrrolidone: BASF Co., Ltd./E8225-17001

Pressure Blowing Concentrator: Hangzhou Aosheng Instrument Co., Ltd./MD200

### Example 1

In 20 mL NMP (N-methyl pyrrolidone) were added 1 g neostigmine, 8 g PLGA (poly (lactic-co-glycolic acid)), and 0.5 g tributyl citrate. The mixture was stirred with cantilever stirrer for 8 hours to give a homogeneous mixture. The homogeneous mixture was placed in a cartridge of a 3D printer.

The designed shape parameters of the polymeric micro-studs were inputted on the computer. The printing parameters were as follows: size of syringe pump extruder: 150 μm; print mode: parallel; height of layer: 0.2 mm; speed of movement: 2 mm/s; temperature of platform: −15° C.

Micro-studs were printed with syringe-pump-based 3D printers. The micro-studs were lyophilized in a lyophilizer for 24 hours and dried in vacuo for 6 hours to give the desired micro-studs.

### Example 2

In 20 mL glacial acetic acid were added 1 g Galantamine, 6 g PLGA (poly(lactic-co-glycolic acid)), 2 g PLA (polylactic acid), and 0.5 g triethyl citrate. The mixture was stirred with a cantilever stirrer for 8 hours to give a homogeneous mixture (Part A).

In 20 mL NMP (N-methyl pyrrolidone) were added 6 g hypromellose, 4 g PLGA (poly (lactic-co-glycolic acid)), and 0.5 g triethyl citrate. The mixture was stirred with cantilever stirrer for 8 hours to give a homogeneous mixture (Part B).

The designed shape parameters of the polymeric micro-studs were inputted on the computer. The printing parameters were as follows: size of syringe pump extruder: 150 μm; print mode: parallel; height of layer: 0.2 mm; speed of movement: 2 mm/s; temperature of platform: −15° C.

The lubrication part was printed with part B as the bottoms of the micro-studs with syringe-pump-based 3D printers. The drug-loading part was printed with part A as the heads of the micro-studs with syringe-pump-based 3D printers. The micro-studs were lyophilized in a lyophilizer for 24 hours and dried in vacuo for 6 hours to give the desired micro-studs.

### Example 3

1 g pyridostigmine, 8 g PLGA (poly (lactic-co-glycolic acid)), and 2 g glycerinum were weighed and then mixed. The mixture was put into a hot melt extruder and extruded to give thermoplastic filaments. The temperature of extruder was 150°. The diameter of the thermoplastic filament was 1.75 mm.

The designed shape parameters of the polymeric micro-studs were inputted on the computer. The thermoplastic filaments were printed into micro-studs with 3D printer. The printing parameters were as follows: infill: 30%; number of shells: 2; height of layer: 0.2 mm; temperature of extruder: 200° C.; speed of movement: 20 mm/s; temperature of platform: 75° C.

### Example 4

Holes were made in glass by chemical etching through holes on a photoresist film patterned by photolithography. Uncured PDMS (polydimethylsiloxane) was poured on this side and cured overnight to give a male PDMS molding. The male PDMS molding was sputter-coated with 100 nm of gold to prevent adhesion with a second PDMS layer cured onto the male molding to create a female PDMS replicate-mold.

In 20 mL dichloromethane were added 1 g neostigmine, 8 g hyaluronic acid, and 0.5 g tributyl citrate. The mixture was stirred with cantilever stirrer for 8 hours to give a homogeneous mixture. The mixture was poured on the PDMS molding in vacuo to give micro-studs. The micro-studs were dried in vacuo for 6 hours and separated from the mold.

### Example 5

Holes were made in glass by chemical etching through holes on a photoresist film patterned by photolithography. Uncured PDMS (polydimethylsiloxane) was poured on this side and cured overnight to give a male

PDMS molding. The male PDMS molding was sputter-coated with 100 nm of gold to prevent adhesion with a second PDMS layer cured onto the male molding to create a female PDMS replicate-mold.

Drug-loaded hydrogel: 5 g ultra-low viscosity carboxymethylcellulose and 5 g PLA were weighed and dissolved in 15 mL purified water to form a viscous hydrogel. 0.5 g ambenonium chloride was weighed and dissolved in the viscous hydrogel.

Pure hydrogel: 5 g ultra-low viscosity carboxymethylcellulose and 5 g PLA were weighed and dissolved in 15 mL purified water to form a viscous hydrogel. Hydrogel was placed on the female PDMS mold in a conical centrifuge tube and centrifuged at 10,000 rpm for 1 h to fill the mold cavities, and was dried at 80° C. for 30 minutes.

Residual hydrogel on the surface of the mold was removed with dry tissue paper, and pure hydrogel without drug was then applied and dried at 80° C. onto the mold to form the backing layer.

### Example 6

Holes were made in glass by chemical etching through holes on a photoresist film patterned by photolithography. Uncured PDMS (polydimethylsiloxane) was poured on this side and cured overnight to give a male PDMS molding. The male PDMS molding was sputter-coated with 100 nm of gold to prevent adhesion with a second PDMS layer cured onto the male molding to create a female PDMS replicate-mold.

Drug-loaded molten material: 0.8 g demecarium bromide, 7 g hypromellose E 5 and 3 g PCL were weighed, mixed and heated until molten.

Drug-loaded molten material was injected into the female PDMS mold at a fixed velocity of 0.40 in/sec (1.016 cm/sec), and pressed at a fixed pressure of 10,000 psi (68.9 MPa) to force the molten material into female PDMS mold. A sheet was applied onto the female PDMS mold, and the female PDMS mold was cooled to form the micro-studs.

### Example 7

Preparation of PDMS mold: PDMS (polydimethylsiloxane) solution was poured into a copper mold, and cured overnight to give a PDMS mold.

Preparation of drug-loading solution: 0.1 g neostigmine bromide, 0.5 g PLGA (poly (lactic-co-glycolic acid)) were added into 5 mL acetone-ethanol solution to give solution A. Solution A was vortexed until it became homogeneous to give solution B. Solution B was concentrated under nitrogen to give solution C.

Preparation of lubrication solution: 1.0 g ACRYPOL 971P was dissolved in 49.0 mL deionized (DI) water. The solution was stirred for 4 hours at a speed of 800 rpm to give solution D.

Preparation of gel solution: 10 g polyvinyl pyrrolidone was dispersed into 40 mL ethanol. The dispersion was stirred for 2 hours at a speed of 800 rpm to give solution E.

Preparation of Micro-Studs:

a. A PDMS micro-stud mold was cast with solution C and centrifuged for 15 minutes at a speed of 4,000 rpm.

b. The remaining solution on the mold surface was scraped off with a metal plate. The mold was centrifuged for 15 minutes at a speed of 4,000 rpm to give the drug-loading part.

c. The mold comprising the drug-loading part was dried at the room temperature for 4 hours, and then was dried at 50° C. for 12 hours.

d. Solution D was poured into the mold of step c. The mold was dried at the room temperature for 8 hours to give the lubrication part.

e. Solution E was poured into the mold of step d. The mold was dried at the room temperature for 12 hours to give the backing layer (soluble carrier material).

### Example 8

Preparation of PDMS mold: PDMS (polydimethylsiloxane) solution was poured into a copper mold, and cured overnight to give a PDMS mold.

Preparation of drug-loading solution: 0.1 g neostigmine bromide, 0.5 g PCL (polycaprolactone) were added into 5 mL trichloromethane to give solution A. Solution A was vortexed until it became homogeneous to give solution B. Solution B was concentrated under nitrogen to give solution C.

Preparation of lubrication solution: 1.0 g ACRYPOL 971P was dissolved in 49.0 mL deionized (DI) water. The solution was stirred for 4 hours at a speed of 800 rpm to give solution D.

Preparation of gel solution: 10 g polyvinyl pyrrolidone was dispersed into 40 mL ethanol. The dispersion was stirred for 2 hours at a speed of 800 rpm to give solution E.

Preparation of Micro-Studs: a. A PDMS micro-stud mold was cast with solution C, and centrifuged for 15 minutes with a speed of 4,000 rpm.

b. The remaining solution on the mold surface was scraped off with a metal plate. The mold was centrifuged for 15 minutes at a speed of 4,000 rpm to give the drug-loading part.

c. The mold comprising the drug-loading part was dried at the room temperature for 4 hours, and the drug-loading part was dried at 50° C. for 12 hours.

d. Solution D was poured into the mold of step c. The mold was dried at the room temperature for 8 hours to give the lubrication part.

e. Solution E was poured into the mold of step d. The mold was dried at the room temperature for 12 hours to give the backing layer (soluble carrier material).

### Example 9

Preparation of PDMS mold: PDMS (polydimethylsiloxane) solution was poured into a copper mold, and cured overnight to give a PDMS mold.

Preparation of drug-loading solution: 0.1 g neostigmine bromide, 0.5 g PLA (polylactic acid) were added into 5 mL trichloromethane to give solution A. Solution A was vortexed until it became homogeneous to give solution B. Solution B was concentrated under nitrogen to give solution C.

Preparation of lubrication solution: 1.0 g ACRYPOL 971P was dissolved in 49.0 mL deionized (DI) water. The solution was stirred for 4 hours at a speed of 800 rpm to give solution D.

Preparation of gel solution: 10 g polyvinyl pyrrolidone was dispersed into 40 mL ethanol. The dispersion was stirred for 2 hours at a speed of 800 rpm to give solution E.

Preparation of Micro-Studs:

a. A PDMS micro-stud mold was cast with solution C, and centrifuged for 15 minutes at a speed of 4,000 rpm.

b. The remaining solution on the mold surface was scraped off with a metal plate. The mold was centrifuged for 15 minutes at a speed of 4,000 rpm to give the drug-loading part.

c. The mold comprising the drug-loading part was dried at the room temperature for 4 hours, and then was dried at 50° C. for 12 hours.

d. Solution D was poured onto the mold of step c. The mold was dried at the room temperature for 8 hours to give the lubrication part.

e. Solution E was poured into the mold of step d. The mold was dried at the room temperature for 12 hours to give the backing layer (soluble carrier material).

### Example 10

Preparation of PDMS mold: PDMS (polydimethylsiloxane) solution was poured into a copper mold, and cured overnight to give a PDMS mold.

Preparation of drug-loading solution: 0.1 g neostigmine bromide was added into 3 mL trichloromethane to give solution A. 1.0 g SAIB (sucrose acetate isobutyrate) was added into 2 mL acetone-ethanol solution to give solution B. Solution A and solution B were mixed and vortexed until it became homogeneous to give solution C. Solution C was concentrated under nitrogen to give solution D.

Preparation of lubrication solution: 1.0 g ACRYPOL 971P was dissolved in 49.0 mL deionized (DI) water. The solution was stirred for 4 hours at a speed of 800 rpm to give solution E.

Preparation of gel solution: 10 g polyvinyl pyrrolidone was dispersed into 40 mL ethanol. The dispersion was stirred for 2 hours at a speed of 800 rpm to give solution F.

Preparation of Micro-Stud:

a. A PDMS micro-stud mold was cast with solution D, and centrifuged for 15 minutes at a speed of 4,000 rpm.

b. The remaining solution on the mold surface was scraped off with a metal plate. The mold was centrifuged for 15 minutes at a speed of 4,000 rpm to give the drug-loading part.

c. The mold comprising the drug-loading part was dried at the room temperature for 4 hours, and then was dried at 50° C. for 12 hours.

d. Solution E was poured into the mold of step c. The mold was dried at the room temperature for 8 hours to give the lubrication part.

e. Solution F was poured into the mold of step d. The mold was dried at the room temperature for 12 hours to give the backing layer (soluble carrier material).

### Example 11

Preparation of PDMS mold: PDMS (polydimethylsiloxane) solution was poured into a copper mold, and cured overnight to give a PDMS mold.

Preparation of drug-loading solution: 0.1 g neostigmine and 0.5 g PLGA (poly (lactic-co-glycolic acid)) were added into 5 mL acetone-ethanol solution to give solution A. Solution A was vortexed until it became homogeneous to give solution B. Solution B was concentrated under nitrogen to give solution C.

Preparation of lubrication solution: 6.7 g hyaluronic acid was dissolved in 10 g deionized (DI) water. The solution was stirred for 4 hours at a speed of 800 rpm to give solution D.

Preparation of gel solution: 10 g polyvinyl pyrrolidone was dispersed into 40 mL ethanol. The dispersion was stirred for 2 hours at a speed of 800 rpm to give solution E.

Preparation of Micro-Stud:

a. A PDMS micro-stud mold was cast with solution C, and centrifuged for 15 minutes at a speed of 4,000 rpm.

b. The remaining solution on the mold surface was scraped off with a metal plate. The mold was centrifuged for 15 minutes at a speed of 4,000 rpm to give the drug-loading part.

c. The mold comprising the drug-loading part was dried at the room temperature for 4 hours, and then was dried at 50° C. for 12 hours.

d. Solution D was poured into the mold of step c. The mold was dried at the room temperature for 8 hours to give the lubrication part.

e. Solution E was poured into the mold of step d. The mold was dried at the room temperature for 12 hours to give the backing layer (soluble carrier material).

### Example 12

Preparation of PDMS mold: PDMS (polydimethylsiloxane) solution was poured into a copper mold, and cured overnight to give a PDMS mold.

Preparation of drug-loading solution: 0.1 g neostigmine bromide and 0.5 g PLA (polylactic acid) were added into 5 mL trichloromethane to give solution A. Solution A was vortexed until it became homogeneous to give solution B. Solution B was concentrated under nitrogen to give solution C.

Preparation of lubrication solution: 6.7 g hyaluronic acid was dissolved in 10 g deionized (DI) water. The solution was stirred for 4 hours at a speed of 800 rpm to give solution D.

Preparation of gel solution: 10 g polyvinyl pyrrolidone was dispersed into 40 mL ethanol. The dispersion was stirred for 2 hours at a speed of 800 rpm to give solution E.

Preparation of Micro-Studs:

a. A PDMS micro-stud mold was cast with solution C, and centrifuged for 15 minutes at a speed of 4,000 rpm.

b. The remaining solution on the mold surface was scraped off with a metal plate. The mold was centrifuged for 15 minutes at a speed of 4,000 rpm to give the drug-loading part.

c. The mold comprising the drug-loading part was dried at the room temperature for 4 hours, and then was dried at 50° C. for 12 hours.

d. Solution D was poured into the mold of step c. The mold was dried at the room temperature for 8 hours to give the lubrication part.

e. Solution E was poured into the mold of step d. The mold was dried at the room temperature for 12 hours to give the backing layer (soluble carrier material).

### Example 13

Preparation of PDMS mold: PDMS (polydimethylsiloxane) solution was poured into a copper mold, and cured overnight to give a PDMS mold.

Preparation of gel solution: 10 g polyvinyl pyrrolidone was dispersed into 40 mL ethanol for 2 hours. The dispersion was stirred at a speed of 800 rpm to give solution A.

Preparation of Backing Layer:

a. A PDMS micro-stud mold was cast with solution A and vibrated to degas.

b. The mold of step a was dried at the room temperature for 8 hours to give the backing layer (soluble carrier material).

### Example 14

The backing layer of Example 13 was put into a test tube with a certain amount of purified water and vortexed for 30 seconds with stirring at a speed of 500 rpm. The backing layer was picked up and put on a table. Half area of the backing layer (area A) was placed outside the edge of table. Polytetrafluoroethylene/silica gel gaskets were added gradually onto the area A until the area A cannot bear the weight of polytetrafluoroethylene/silica gel gaskets. Polytetrafluoroethylene/silica gel gaskets were collected and weighed to evaluate softening effects of the backing layer.

| | Weight of polytetrafluoroethylene/ |
|---|---|
| Amount of purified water | silica gel gaskets |
| 40 μL | 83.475 mg |
| 30 μL | 125.550 mg |
| 20 μL | 165.457 mg |
| 10 μL | 320.432 mg |

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

What is claimed is:

1. An ophthalmic micro-stud formulation, comprising a drug-loading part as a head of the micro-stud, a lubrication part as a bottom of the micro-stud, and a soluble carrier material, wherein the lubrication part is attached to the soluble carrier material.

2. The ophthalmic micro-stud formulation of claim 1, wherein the lubrication part is a drug-free part.

3. The ophthalmic micro-stud formulation of claim 1, wherein the lubrication part comprises a gel material, hyaluronic acid (HA) or a mixture thereof.

4. The ophthalmic micro-stud formulation of claim 3, wherein the gel material is selected from the group consisting of carbomer, poloxamer, calcium polycarbophil, polyethylene oxide, polyethylene glycol and a mixture thereof.

5. The ophthalmic micro-stud formulation of claim 1, wherein the soluble carrier material comprises polyvinyl pyrrolidone.

6. The ophthalmic micro-stud formulation of claim 1, wherein the drug-loading part comprises an active pharmaceutical ingredient (API), a biodegradable material and a pharmaceutically acceptable excipient.

7. The ophthalmic micro-stud formulation of claim 6, wherein the biodegradable material is selected from the group consisting of PLGA (poly(lactic-co-glycolic acid)), PLA (polylactic acid), PLC (polylactide-caprolactone copolymer), PGA (polyglycolic acid), hyaluronic acid, collagen, SAIB (sucrose acetate isobutyrate), poly(orthoesters), PEG (polyethylene glycol), alginate, PCL (polycaprolactone), PCE (polycaprolactone-polyethylene glycol), PCEL (polycaprolactone-polyethylene glycol-polylactide), PHB (poly-β-hydroxybutyrate) and a mixture thereof.

8. The ophthalmic micro-stud formulation of claim 1, wherein the ophthalmic micro-stud formulation has one micro-stud or an array of micro-studs.

9. The ophthalmic micro-stud formulation of claim 8, wherein the micro-stud is elevated cylindrical, cone-shaped, cube-like, or rectangle-like.

10. The ophthalmic micro-stud formulation of claim 1, wherein the carrier material has a long diameter of not more than 22 mm.

11. The ophthalmic micro-stud formulation of claim 1, wherein the carrier material has a short diameter of not more than 4 mm.

12. The ophthalmic micro-stud formulation of claim 1, wherein the height of the micro-stud is not more than 2 mm.

13. The ophthalmic micro-stud formulation of claim 1, wherein the width of the micro-stud is not more than 2 mm.

14. The ophthalmic micro-stud formulation of claim 1, wherein the micro-stud is hollow.

15. The ophthalmic micro-stud formulation of claim 6, wherein the active pharmaceutical ingredient (API) comprises a first active pharmaceutical ingredient and a second active pharmaceutical ingredient.

16. The ophthalmic micro-stud formulation of claim 15, wherein the first active pharmaceutical ingredient is selected from the group consisting of neostigmine bromide, neostigmine, pyridostigmine, edrophonium chloride, ambenonium chloride, physostigmine, demecarium bromide and galantamine.

17. The ophthalmic micro-stud formulation of claim 15, wherein the second active pharmaceutical ingredient is selected from the group consisting of hemostatics and coagulants.

18. A process for preparing an ophthalmic micro-stud formulation, comprising preparing a micro-stud via 3DP (three dimensional printing), mold-based hot embossing, injection molding, mold-based centrifuging, mold-based vacuum, injection molding, mold-based photopolymerization, droplet-born air blowing, or stretching photolithography, wherein the ophthalmic micro-stud formulation comprises a drug-loading part as a head of the micro-stud, a lubrication part as a bottom of the micro-stud, and a soluble carrier material, and the lubrication part is attached to the soluble carrier material.

19. The process of claim 18, wherein the 3DP (three dimensional printing) is selected from the group consisting of fused deposition modelling, direct metal laser-sintering, electron beam melting, selective laser sintering, selective laser melting, selective heat sintering, stereo lithography appearance, digital light processing, polyjet, multi-jet printing, continuous liquid interface production, two-photon polymerization, 3DP (three dimensional printing) and gluing, binder jetting, color jet printing, nanoparticle jetting, laminated object manufacturing, laser engineered net shaping, multi jet fusion, plaster-based 3DP (three dimensional printing), laser cladding forming, and syringe-pump-based 3DP (three dimensional printing).

20. A method for treating and preventing oculopathy, comprising administering an ophthalmic micro-stud formulation to a palpebral conjunctiva superior to a superior tarsal border of an affected eye of a subject in need thereof, wherein the ophthalmic micro-stud formulation comprises a drug-loading part as a head of the micro-stud, a lubrication part as a bottom of the micro-stud, and a soluble carrier material, and the lubrication part is attached to the soluble carrier material.

21. The method of claim 20, wherein the oculopathy is selected from the group consisting of ocular myasthenia gravis (OMG), blepharospasm, dermatolysis palpebrarum, involutional, myogenic, neurogenic, and congenital ptosis, trichiasis and eyelid tumors.

22. The method of claim 20, wherein the method comprises everting an upper eyelid to expose a palpebral conjunctiva.
